(19) United States
(12) Patent Application Publication
(10) Pub. No.: US 2024/0108864 A1
(43) Pub. Date: Apr. 4, 2024

(54) DRUG-COATED BALLOON DEVICE, SYSTEM, AND PROCEDURE

(71) Applicant: CANON U.S.A., INC., MELVILLE, NY (US)

(72) Inventors: Seiji Takeuchi, Newton, MA (US); Mie Kunio, Yokohama (JP)

(21) Appl. No.: 17/937,157

(22) Filed: Sep. 30, 2022

(51) Int. Cl. A61M 25/10; A61B 1/04; A61B 5/00; A61B 8/12; A61K 45/06

(52) U.S. Cl. CPC A61M 25/10; A61B 1/043; A61B 5/0066; A61B 5/0071; A61B 5/6853; A61B 8/12; A61K 45/06; A61B 2562/0247; A61M 2025/105

(57) ABSTRACT

There is provided medical devices and methods of use. The medical device comprising: an elongated tube; a balloon disposed over the elongate tube having at least one drug and at least one fluorescent agent on an outer surface of the balloon; an optical probe at the distal end of the elongate tube comprising an optical fiber configured to guide illumination light coming from a light source and an optical member configured for fluorescence imaging; and one or more detectors configured for fluorescence detection. The probe may comprise an optical probe for fluorescence imaging, and optionally an additional probe component for structural imaging or physiological sensing. The method can be particularly useful for determining whether sufficient dose of a drug has been transferred from a balloon to the lumen.

### FIELD OF THE INVENTION

The present disclosure relates generally to drug coated medical devices such as drug coated balloon catheters and optical imaging such as OCT, fluoresce, IVUS, and the like, and methods of diagnosis and treatment using drug coated medical devices and optical imaging.

### BACKGROUND OF THE INVENTION

Although introduction of drug-eluting stent (DES) in coronary artery disease improved the rate of in-stent restenosis compared to that when using bare metal stent (BMS), it is still occurs in 3 to 20% of patients and remains to be an issue after treatment. (Dangas et al., JACC, 2010; 56(23):1897-907) In DAEDALUS study, drug-coated balloon (DCB) showed its effectiveness and safety for in-stent restenosis in bare metal stent, while it showed its safety but not effectiveness for in-stent restenosis in drug eluting stent. (Giacoppo, et al., JACC, 2020; 75(21): 2664-2678)

Drug coating balloons have been developed as treatment options for cardiovascular disease, other peripheral artery diseases, and diseases involving non-vascular lumens such as, for example, asthma, chronic obstructive pulmonary disease (COPD), prostate cancer, benign prostatic hyperplasia and strictures in the urethra, esophagus, or sinus. Drug coated balloon catheters include those describe in U.S. Pat. No. 10,058,636 (catheter coated with Paclitaxel), U.S. Pat. No. 9,295,663 (catheter with Sirolimus), U.S. Pat. No. 10,668,188 (catheter with anti-inflammatory or anti-proliferative agents), and U.S. Pat. No. 10,987,451 (catheter coated with Paclitaxel).

There is a need to confirm whether effective transfer of drug from balloon surface to the vessel has occurred or not. There has been a concern of flaking of coating from balloon, which has been a major concern as the risk of thrombus. The drug coating lost in transit to vessel is a problem with DCB where 10% of drug may be lost before the target lesion is reached and 10% remain on the balloon after withdrawal. (Speck, U et al., Cardiovasc Intervent Radiol. 2018 October; 41(10):1599-1610.)

The method available to confirm proper treatment procedure of drug coated balloon has been to see if the balloon has dilated the stenosis lesion properly. This can be done by viewing the region using angiography, IVUS, and/or OCT or by confirming either the blood flow or physical opening of the vessel using FFR or angiography. However, each of these are useful for determining vessel dilation, not drug transfer. Accordingly, it would be desirable to provide a method for confirming the success or amount of drug transferred when using a drug coated balloon procedure.

### SUMMARY OF THE INVENTION

Accordingly, it is an object of the present disclosure to provide medical devices and methods of use. In some embodiments, the medical device comprises an elongated tube; a balloon disposed over the elongate tube having at least one drug and at least one fluorescent agent on an outer surface of the balloon; an optical probe at the distal end of the elongate tube comprising an optical fiber configured to guide illumination light coming from a light source and an optical member configured for fluorescence imaging; and one or more detectors configured for fluorescence detection.

In some embodiments, the medical device comprises an elongated tube; a balloon disposed over the elongate tube having at least one drug and at least one fluorescent agent on an outer surface of the balloon; a probe at the distal end of the elongate tube, a first detector configured for fluorescence detection; and a second detector configured for detection of the structural imaging or physiological sensing. The probe of this embodiment is configured for fluorescence imaging, and for structural imaging or physiological sensing.

Methods of analysis using these medical devices are also contemplated. One such method comprises: inserting a catheter comprising an optical probe and a drug-coated balloon into a lumen, wherein the optical probe comprises an optical fiber configured to guide illumination light coming from a light source and an optical member configured for fluorescence imaging; and wherein the drug-coated balloon has a drug and a fluorescent agent on the outer surface of the balloon; expanding the drug-coated balloon in a region of the lumen to create a drug-transfer region of the lumen; detecting a fluorescence signal from the fluorescent agent in the drug-transfer region with the optical probe; correlating the fluorescence signal with a concentration of the drug transferred from the drug-coated balloon; and displaying information based on the concentration of the transferred drug.

In some of the medical devices and/or methods as described above, the structural imaging is optical coherence tomography (OCT) imaging or intravascular ultrasound (IVUS) imaging and/or the physiological sensing is fractional flow reserve FFR) and the second detector is configured for detecting pressure.

In some embodiments, the method provides for imaging at least a portion of the drug-transfer region of the lumen with the optical probe to obtain an image; and confirming or verifying dilation of the lumen and/or transfer of drug in the drug-transfer region with the image. The correlation of the fluorescence signal with a concentration of the drug may be uses a correlation factor obtained from ex vivo studies.

In some embodiments, the information based on the concentration of the transferred drug is information that compares the concentration of the drug transferred to a dose of the drug known to be sufficient for a treatment.

Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIGS. 1(A), 1(B), and 1(C) are diagrams showing an embodiment of a system as described herein. FIG. 1(A) depicts a balloon catheter in expanded form. FIG. 1(B) depicts a balloon catheter not expanded. FIG. 1(C) is a cartoon depiction of a section of the catheter in FIG. 1(A) illustrating the presence of drug and fluorophore.

FIGS. 2(A) and 2(B) are diagrams showing at least one embodiment of a system which comprises an imaging catheter (FIG. 2(A)) and an imaging catheter with a balloon (FIG. 2(B)) in accordance with one or more aspects of the present disclosure.

FIG. 3 is a diagram showing an embodiment of a system as described herein, in accordance with one or more aspects of the present disclosure.

FIG. 4 is a diagram showing an embodiment of a system as described herein.

FIG. 5 is a workflow using the methods and systems of the present embodiments.

FIG. 6 is a diagram showing an embodiment of a system as described herein.

FIG. 7 is a work flow using the methods and systems of the present embodiments.

FIG. 8 is an exemplary system workflow.

FIGS. 9(A)-9(D) are diagrams showing the under expansion (FIGS. 9(A) and 9(B)) and the appropriate expansion (FIGS. 9(C) and 9(D)).

FIG. 10 is a diagram showing a computer system.

### DETAILED DESCRIPTION OF THE PRESENT INVENTION

### Balloon Catheters

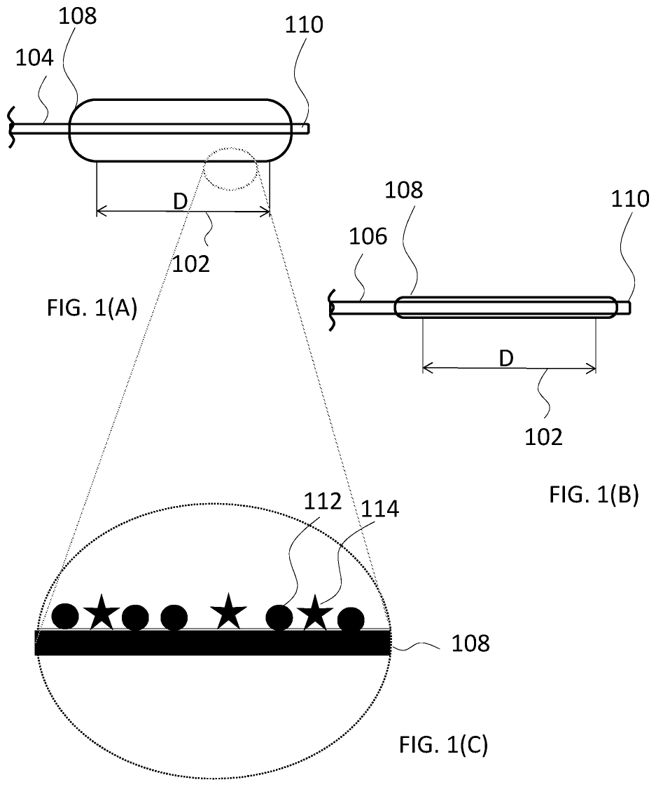

There are a number of balloon catheters for the expansion of a lumen and drug delivery on the market and in research. Any suitable balloon catheter to one of ordinary skill in the art may be used. The balloon catheter may include an inflatable balloon at the distal end of the catheter. FIG. 1(A) shows a balloon catheter with the balloon expanded. The arrow 102 shows the region on the catheter where dilatation of the lumen is performed, the length of the ballooning section being D. The central catheter portion 104 extents past the balloon 108 to an atraumatic tip 106 extends beyond the drug coated balloon 108. At the atraumatic tip 106, a rapid exchange guide for insertion on a guidewire and/or a radio-opaque marker may also be included (not shown). In FIG. 1(B), the balloon 108 is collapsed around the catheter portion 104. FIG. 1(C) is an expansion of the balloon 108 of FIG. 1(A) where the outer coating including one or more drug 112 and one or more fluorescent agent 114 can be seen on the surface of the balloon 108.

The drug is any therapeutic agent that may be administered by use of a balloon catheter. The drug may be an agent for the prevention and/or treatment of stenosis such as Sirolimus or Paclitaxel. More broadly, the drug may be therapeutic agent for prophylaxis or treatment of a vascular or nonvascular body lumen, such as with an anti-inflammatory or anti-proliferative drug. Additional additives for drug affinity, solvation agents, oils, lipids, etc. may also be included with the drug. An exemplary list of drugs is provided, for example, in U.S. Pat. No. 10,668,188.

The coating may also contain one or more excipients or carriers. An excipient may be preferentially added to aid in the transfer of the drug from the surface of the balloon to the tissue. The carrier can be selected for the speed and concentration of drug delivery from the balloon to the tissue in the lumen. Excipients, such as bio-degradable polymers, urea, and shellac into or onto which the drug is embedded can be used to optimize the release kinetics of the drug into the arterial tissue. Concentration of drug delivery may mean how much of drug is transferred to a unit area or the drug dose transferred to a predetermined area of the inner lumen where the balloon expands and touches.

The fluorescent agent 114 is any compound that fluoresces and is biocompatible. The terms dye, fluorescence dye, and fluorophore may be used instead of fluorescent agent. Examples of fluorescent agents are the Cy dyes, Alexa Fluor dyes, methylene blue and indocyanine green (ICG). There have been studies of uses of fluorescent agents for characterization of vessel tissue and one or more of these dyes are used. Some of the useful dyes include the following:

TABLE 1
Fluorescence Dyes and their excitation and emission wavelengths.

| | Excitation | Emission (Detection) |
|---|---|---|
| Fluorescence dyes | wavelength [nm] | wavelength [nm] |
| Cy2 | 489 | 505 |
| Cy3 | 552 | 565 |
| Cy3.5 | 581 | 596 |
| Alexa Fluor 350 | 346 | 442 |
| Alexa Fluor 405 | 401 | 421 |
| Alexa Fluor 488 | 495 | 519 |
| Alexa Fluor 532 | 532 | 554 |
| Alexa Fluor 546 | 556 | 573 |

In some embodiments, the drug and fluorescent agent are co-encapsulated into a liposome, nanoparticle, or other moiety for controlled delivery. For example, sirolimus (rapamycin) and indocyanine green (ICG) were co-encapsulated into folate targeted thermosensitive liposomes. This was used to enhance tumor therapeutic and diagnostic functions. (See, for example, Pang X, Wang J, Tan X, et al. ACS Appl Mater Interfaces. 2016; 8:13819-13829.

In other embodiments, the drug and fluorescent agent are linked together through a linker, such a small molecule or peptide or a cleavable linker such as a self-immolative linker or a peptide-enabled linker. This enhances the effectiveness of the fluorescent agent as an indicator of drug transfer to the tissue since a proportional amount of dye is in contact with the drug moiety. (See, for Example, Lang et al., Journal of Pharmaceutical Analysis 10 (2020) 434-443). There are various drug-dye conjugates that may be used in conjunction with the present invention. Additionally, known conjugation chemistry may be adapted from some therapeutic modalities to accommodate the drug moieties used with balloon delivery.

In yet other embodiments the fluorescent agent may be included as part of a quenched complex, where two dyes are linked in such proximity that the fluorescence from them is quenched until they are separated (such as by an enzymatic cleavage in the region of interest).

The coating may be a direct coating on the balloon material. For example, the drug and dye may be dissolved in a carrier that is drop coated or dip coated onto the balloon surface and the carrier allowed to evaporate. The coating may be coating on a highly elastic wrap that is placed around the balloon (See, for example, Torsten, H, et al., European Cardiology 2010:6(4):40-4.)

Preferably, the coating will coat the balloon substantially evenly over the surface of the balloon that comes in contact with tissue upon inflation. As used herein, the term “substantially” is meant to allow for deviations from the descriptor that do not negatively affect the intended purpose.

### Image Catheters

The balloon catheter above contemplates the balloon inflation and drug delivery aspect of the present invention. An imaging catheter, either as a separate catheter or combined with the balloon, is also used in the present invention.

Imaging catheters and endoscopes have been developed to access to internal organs. In order to acquire cross-sectional images of tubes and cavities such as vessels, esophagus and nasal cavity, the imaging probe can be rotated in the lumen. In addition, the imaging probe can be simultaneously translated longitudinally during the rotation so that helical scanning pattern images are obtained and information along a longitudinal portion of the lumen is obtained. This translation is most commonly performed by pulling probe back towards proximal end and therefore referred to as a pullback.

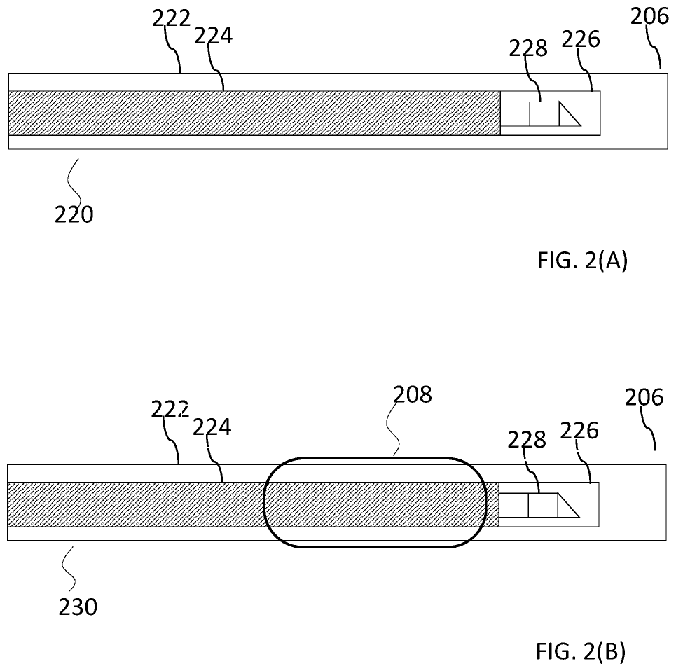

FIG. 2(A) shows an embodiment of an imaging catheter 220 having an atraumatic tip 206 including a sheath 222, a coil 224, and a protector 226. At the distal end of the catheter, there is an optical probe 228, which is proximal to the atraumatic tip 206. A rapid exchange guide for insertion on a guidewire and/or a radio-opaque marker may also be included at the distal end of the catheter (not shown). The coil 224 delivers torque from a proximal end to a distal end thereof (e.g., via or by a rotational motor in a patient interface unit). In one or more embodiments, the coil 224 is fixed with/to the optical probe 228 so that a distal tip of the optical probe 228 also spins to see an omnidirectional view of a lumen. For example, fiber optic catheters and endoscopes may reside in the sample arm of an OCT interferometer in order to provide access to a lumen. In addition to the arteries, this system may be used in internal organs, gastro-intestinal tract or any other narrow area, that are difficult to access. As the beam of light passes through the optical probe 228, it is directed out of the catheter 220 (or endoscope) to the lumen wall. Light is also captured by the optical probe 228 and this coherent light from the OCT system and emission light form the fluorescence system is returned via an optical fiber(s) to the detector. The optical probe (also called an imaging core) is rotated via a rotary junction so the light moves across the surface of interest, such that cross-sectional images of one or more samples are obtained. In order to acquire three-dimensional data, the optical probe 228 is simultaneously translated longitudinally during the rotational spin resulting in a helical scanning pattern. This translation is most commonly performed by pulling the tip of the probe 228 back towards the proximal end and therefore referred to as a pullback.

In one or more embodiments, the optical probe (e.g., the probe 228 of the catheter 220) may comprise or include an optical fiber connector, an optical fiber, and an optical member disposed at or near the distal end of the optical probe. The optical member may be a lens. The optical fiber operates to deliver light to the distal lens. The distal lens operates to shape the optical beam and to illuminate light to the lumen and to collect light from the lumen efficiently. The double clad fiber may be used to transmit and/or collect OCT light through the core and to collect Raman and/or fluorescence light from sample (e.g., lumen or tissue) through the clad. The lens may be used for focusing and collecting light to and/or from the sample (e.g., lumen or tissue). The scattered light through the clad may be relatively higher than that through the core in a case or instance where a size of the core is smaller or much smaller than a size of the clad.

FIG. 2(B) shows an imaging catheter 230 a sheath 222, a coil 224, a protector 226 and an optical probe 228. This imaging catheter 230 also has a balloon 208 that, in this embodiment, is located directly behind the optical probe 228 portion of the catheter 230. In other embodiments, the balloon may be located distal to the optical probe portion of the catheter 228, or may be located further proximal on the probe.

### Catheter System

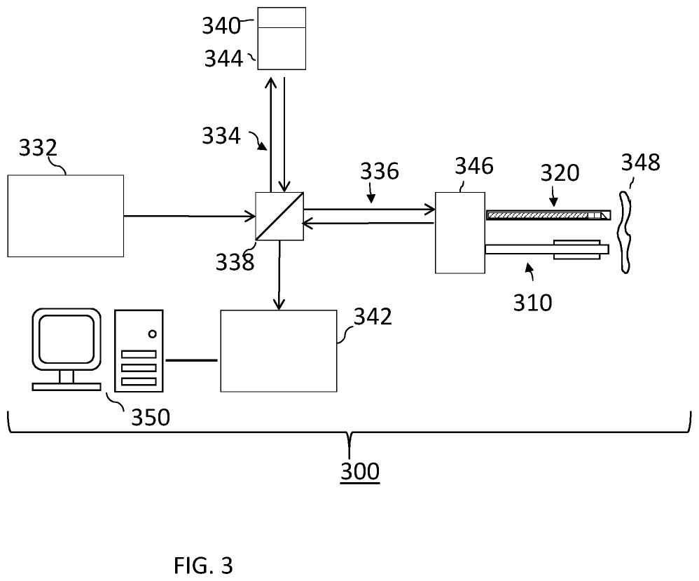

FIG. 3 shows an imaging system 300 which operates to utilize a balloon to deliver a drug and an optical probe for analysis of the drug delivery in accordance with one or more aspects of the present disclosure. The system 300 comprises one or more light sources 332, a reference arm 334, a sample arm 336, a splitter 338 (also referred to herein as a “beam splitter”), a reference mirror (also referred to herein as a “reference reflection”) 340, and one or more detectors 342. The system 300 may include a phase shift device or unit 344. In one or more embodiments, the system 300 may include a patient interface device or unit (“PIU”) 346 and one or more catheters 310 and 320 (as diagrammatically shown in FIGS. 1(A), 1(B), 2(A), and 2(B) above, and may include, for example, catheter 330), and the system 300 may interact with a sample 348 (e.g., via the one or more catheters 310 and 320 and/or 330 and/or the PIU 346). In one or more embodiments, the system 300 includes an interferometer or an interferometer is defined by one or more components of the system 300, such as, but not limited to, at least the light source 332, the reference arm 334, the sample arm 336, the splitter 338 and the reference mirror 340.

For optical coherence tomography (OCT) subsystem, the light source 332 operates to produce a light to the splitter 338, which splits the light from the light source 332 into a reference beam passing into the reference arm 334 and a sample beam passing into the sample arm 336. The beam splitter 338 is positioned or disposed at an angle to the reference mirror 340, the one or more detectors 342 and to the sample 348. The reference beam optionally goes through the phase shift unit 344 (when included in a system, as shown in the system 300), and the reference beam is reflected from the reference mirror 340 in the reference arm 3342 while the sample beam is reflected or scattered from a sample 348 through the PIU (patient interface unit) 346 and the catheter 320 and/or 330 in the sample arm 103. Both of the reference and sample beams combine (or recombine) at the splitter 338 and generate interference patterns. The output of the system 300 and/or the interferometer thereof is continuously acquired with the one or more detectors 342, e.g., such as, but not limited to, photodiodes or multi-array cameras. The one or more detectors 342 measure the interference or interference patterns between the two radiation or light beams that are combined or recombined. In one or more embodiments, the reference and sample beams have traveled different optical path lengths such that a fringe effect is created and is measurable by the one or more detectors 342. Electrical analog signals obtained from the output of the system 300 and/or the interferometer thereof are converted to digital signals to be analyzed with a computer, such as, but not limited to, the computer 350 (shown in FIG. S, respectively, discussed further below). The one or more detectors 342 measures visible and/or infrared light that is a fluorescence signal form the tissue. Optical filters may be used in the detector to detect the specified emission wavelength range for the fluorescent agent(s) 114 selected.

The light source 332 may include a plurality of light sources or may be a single light source. In one or more embodiments, the light source 332 may be a radiation source or a broadband light source that radiates in a broad band of wavelengths. In one or more embodiments, a Fourier analyzer including software and electronics may be used to convert the electrical analog signals into an optical spectrum. The light source 332 may include one or more of a laser, an organic Light-Emitting Diode (OLED), a Light-Emitting Diode (LED), a halogen lamp, an incandescent lamp, supercontinuum light source pumped by a laser, and/or a fluorescent lamp. The light source 332 may be fiber coupled or may be free space coupled to the other components of the system or systems discussed herein, such as, but not limited to, the system 300 or any other system discussed herein, etc.

In one or more embodiments, one of the one or more light sources 332 is an excitation light with a wavelength (e.g., any predetermined wavelength visible to infrared (IR)), for example, 0.633 um from a light source 332 and may be delivered to the tissue to through the catheter 320. The fluorescence light may be collected with the catheter (e.g., the catheter 320 of FIG. 3) and delivered to detectors 342 such as a photo-multiplier tube (PMT) or tubes (PMTs), etc.) via the PIU 346. Other wavelengths, in the visible and NIR are also contemplated. In one or more embodiments, the patient interface unit (PIU; e.g., the PIU 346 as further discussed below) may include or comprise a free space beam combiner so that the excitation light couples into a common DCF or other possible/useful fiber with OCT. The excitation light may be illuminated to the tissue from a distal end of the optical probe in the catheter (e.g., the catheter or the probe 320). The one or more detectors 342 of the fluorescence sub-system may send the signal(s) to a second data acquisition unit that is part of the computer 350 or a separate unit.

In one or more embodiments, the one or more detectors 342 of the OCT sub-system may send the signal(s) to a computer system 350. This computer system 350 may include one or more data acquisition unit(s) or processor(s).

In FIG. 3, the PIU 346 is seen as attached (or attachable to) two distinct catheters—the imaging catheter 320 and a balloon catheter 306. These catheters may be attached sequentially as one and then the other catheter is directed into a lumen during a procedure. Also contemplated are systems where two separate PIU are provided for the two separate catheters. Thus, the imaging catheter will have a PIU where imaging data is obtained and a separate PIU is used to direct the balloon catheter and is able to inflate/deflate the balloon. In yet other embodiments, both catheter functions (imaging and balloon) are provided on a single catheter and PIU. In such embodiments, the second catheter of FIG. 3 is not present. The use of a single catheter is particular advantageous since this simplifies the workflow in that doctor or other clinician does not have to remove the balloon catheter and insert a second catheter. Additionally, the exact location where the balloon expanded and transferred the drug is better assessed when a single catheter is used since the distance between the optical probe and thus location of the fluorescent data is a known, set distance from the balloon location.

DCB with Fluorescent Material in the Coating

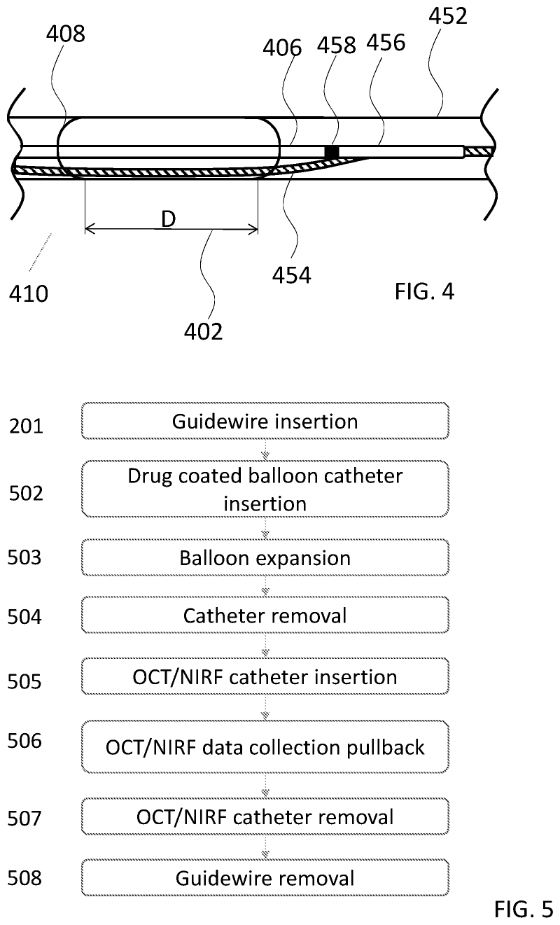

FIG. 4 shows an embodiment of this invention in cardiovascular vessel where a catheter includes a drug coated balloon where the coating contains the drug as well as one or more fluorophores or fluorescent dyes. FIG. 5 shows the example flow of the procedure. In FIG. 4, 452 is the cardiovascular vessel on which percutaneous cardiovascular intervention (PCI) is performed. The arrow, 402, shows the region of the vessel where dilatation is to be performed, the length of the ballooning section being D. For PCI, the guidewire 454 is first inserted through the occluded region of the vessel (Step 501). Then catheter 410 has a rapid exchange guiding tip 456 and radio-opaque marker 458 is inserted on the guidewire (Step 502). The radio-opaque marker at a predetermined distance to the balloon allows the viewing and positioning of the catheter to the region of interest on angiography. The balloon 408 has, on the outside, a coating which includes the drug to avoid restenosis, such as Sirolimus or Paclitaxel. The coating also contains fluorescent dyes, fluorescence agent, or fluorophores, such as a Cy or Alexa Fluor dye, methylene blue or indocyanine green (ICG). There multiple such fluorophores approved for human use. The balloon is expanded to dilate the stenosis region (503), by injecting saline or other fluid through the main catheter lumen 104 to the balloon (Step 503). At this step, the coating on the outer surface of the balloon is transferred by the pressure applied to the vessel wall by the balloon, both for the purpose of expanding the stenosed (occluded) wall and for transfer of the coating including the drug and fluorescence agent. Then the balloon catheter is pulled and removed, riding on the guidewire (Step 504). Although not shown in FIG. 4, OCT/Fluorescence catheter is then inserted (step 505), riding on the guidewire with its own rapid exchange guiding tip. After positioning the catheter properly, with confirmation on angiography of the radio opaque maker at a predetermined distance from imaging position for aligning the imaging position of the catheter to the balloon treated position, imaging pullback is performed (Step 506). In the catheter, the optical probe is pulled back while the probe is spinning with its beam illuminating laterally to the inner wall of the vessel. The OCT light in 1.3 micron wavelength range and near-infrared fluorescence light from the vessel wall are collected through the optical probe, optical signal is detected with detectors and processed in the processing system, which is connected to the probe and catheter. The procedure may be done without the guidewire, when the catheter itself serves to navigate to the location of the stenosis.

After the OCT/Fluorescence pullback step 506, the collected light is analyzed on the system. OCT light signal is processed as OCT data and the system displays the structural state of the vessel, showing the diameter of the vessel at the location of the procedure. The coated balloon was removed in previous step 504. Thus, the only fluorescence agent remaining in the vessel are the natural auto fluorescent material(s) in the vessel wall or the fluorescence agent included in the balloon coating and transferred to the vessel wall.

The fluorescence data is processed and information is displayed to the user. The process may include the distance correction of fluorescence value based on distance from the catheter to the wall from OCT data, as the fluorescence value is dependent on the distance from the source of fluorescence. The fluorescence data may be compared or compensated with correlation factor(s). One way to determine the correlation factor is using a predetermined fluorescence value from, for example, a lookup table that is used to correlate the in vivo fluorescence intensity with concentration of the fluorophore on the vessel wall. In other embodiments, experiments and measurement are performed to determine the coefficient of fluorescence measured with respect to the pressure of the pressing the drug coated material to the tissue. This experiment may be done with optimal drug and fluorescent agent concentration on the material to match the actual drug coated device. Alternatively, there is a separate experiment to measure the fluorescence intensity per given drug concentration with the fluorescence agent already pre-mixed. For example, in the case of the conjugate of fluorescent agent and drug, the fluorescent intensity versus the drug conjugate concentration may be measured. This data may, in turn, be correlated with the drug concentration transferred from the balloon to the vessel wall since the amount of fluorophore transferred from the balloon coating to the vessel wall is proportional to the transfer amount of the drug. Such information may be derived, for example, from an ex vivo study of drug transfer to a lumen or a mouse model of drug concentrations. While this embodiment is demonstrated with OCT, in some other embodiments, fluorescence but no OCT data is obtained during the pullback. The drug transfer can be determined using the fluorescence of the added fluorophore transferred off the balloon, the addition of OCT is useful for the information it provides regarding the physical characteristics of the vessel and is not necessary for determining drug transfer.

DCB Catheter with MMOCT Function

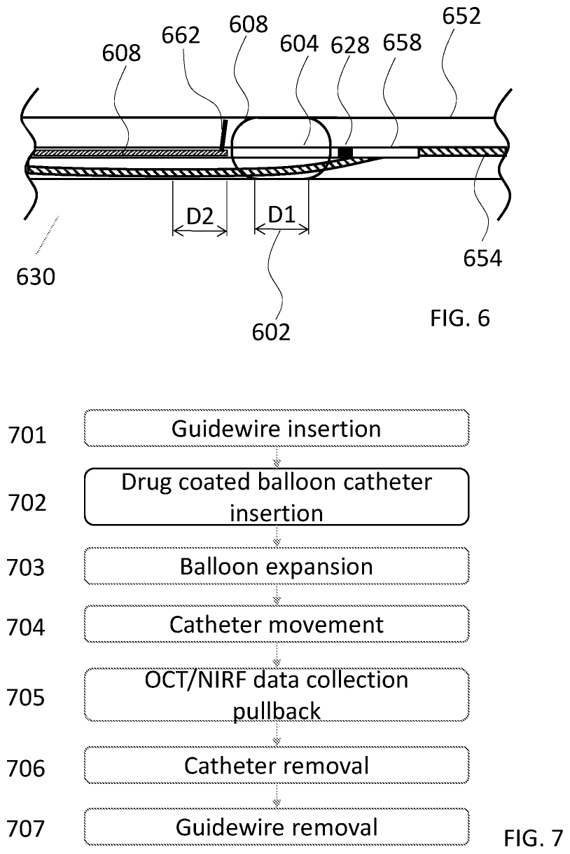

FIG. 6 shows the exemplary configuration for catheter 630 of another embodiment and FIG. 7 shows an example procedure using this device. The vessel to be treated is 652. The guidewire 654 is inserted in the vessel to be treated (Step 701). The drug coated balloon catheter 610 is inserted into the vessel of interest (Step 702). The catheter has the rapid exchange guiding tip 656 and rides on the guidewire. The radio opaque maker 658 is used as marker on the angiography to locate the catheter to the desired location of the vessel. The arrow 302 shows the location of the balloon expansion of the catheter, and for this figure, the balloon expansion length is D1. The outside surface of balloon is coated with drug and fluorescent agent, similar to what was described above. The balloon is expanded by supplying saline or other expanding fluid to the balloon 608 through one of the lumens in the main catheter portion 604 (Step 703). Catheter is moved so that the balloon expanded region of the vessel matches or includes the imaging pullback position D2 (Step 705). The optical probe 628 in the main lumen of the catheter emits light 662 and is pulled back as the probe is spinning and the laser beam or other light source scans the inner wall of the vessel in helical motion. The light includes OCT modality light of, for example, 1.3 micron wavelength range and excitation light for fluorescence modality. The probe collects the reflected/scattered light from the illuminated area and sends the light back to the system for optical signal collection. The OCT light signals is processed to show the dilation of the vessel and fluorescence light detected is processed to show the fluorescence amount from the inner wall which is representative of and proportional to the drug transferred to the lumen wall. The optical probe imaging section is located more proximal than the balloon section in FIG. 6 and this embodiment. However, the imaging window location with respect to the balloon location on the catheter may be inverted to have optical imaging section in the distal end than the balloon.

Spin without Pullback

While the embodiment described in FIG. 6 shows the pullback length of D2, in some embodiments, the OCT/Fluorescence data collection is done without pullback, but only with spinning motion of the optical probe. In step 705, The OCT/Fluorescence data collection performs just spinning detection at a substantially stationary location in the longitudinal direction of the vessel. Since the transfer of drug to the vessel wall should be similar throughout the length of the balloon, fluorescence signal at a single location will give information as to the amount of drug transferred since the data collected will be representative of one cross-section of the balloon dilated portion of the vessel. Before the OCT/fluorescence data is collected, the catheter 630 is moved within the vessel of locations of interest in the longitudinal direction within the dilated region D1 to obtain the representative cross section data of lumen diameter for dilation by OCT and representative cross section data of drug transfer by fluorescence.

### IVUS/NIRF

Another embodiment of the present invention uses IVUS for vessel imaging. As discussed above, IVUS can be used as the structural imaging modality in place of OCT for dilation confirmation after the procedure. On the probe 630 as shown in FIG. 3, in addition to the probe component including optical fiber to deliver excitation light and collect fluorescent light, the probe includes a second component that includes electrical wires to supply electric signals to and from the piezoelectric transducer, located at the tip. Ultrasound waves are emitted (similar to the light emitted at 662 in FIG. 6 is emitted) and detected by the piezoelectric transducer at the tip for ultrasound imaging, while the optical fiber, with its distal optics deliver excitation beam to and collect the fluorescence light from the inner wall of the vessel. There are various configurations of fluorescence and IVUS, where the IVUS beam may be emitted in opposite direction, in orthogonal direction or in the same azimuthal direction with respect to the fluorescence excitation beam in the plane perpendicular to the axis of rotation. The procedure for use is similar to those in FIG. 7, but instead of OCT/Fluorescence catheter in Steps 704 through 706, IVUS/Fluorescence catheter is used.

### FFR/NIRF

Another embodiment of the present invention uses fractional flow reserve (FFR) for vessel dilatation confirmation by pressure measurement. The two pressure sensors are located distal and proximal to the balloon on the main balloon catheter and the pressure drop across the stenosis is measured before and after the balloon procedure. When the pressure drop across the stenosis is larger than 20%, the stenosis needs to be dilated and it is necessary to measure the pressure drop is decreased to confirm the success of balloon expansion after the procedure. (See George J. et al., Curr Cardiol Rev. 2015 August; 11(3): 209-219.)

### Wavelength Separation for Tissue Characterization and Drug Coating Transfer

Vascular tissue which has damages, inflammations, or disease shows auto-fluorescence, without any external fluorophores added. (See Hongki Yoo, et al. Nature Medicine volume 17, pages 1680-1684 (2011)). Thus, this information can be combined with the fluorescence signals as provided herein to include information about both the inflammatory or disease properties of the tissue and the transfer of drug to the same or similar tissue location. See, for example, U.S. Pat. Pub. 10,912,462, 10,952,616, 11,147,453, 10,674,985, and U.S. Pat. Pub. 2021/0407098. Thus, in some embodiments, two different fluorescence signals are separated based on spectra or wavelength range. The fluorescence tissue characterization based on auto fluorescence is done, for example, using 635 nm as excitation light and emitted light with a range between 650 and 900 nm as detected fluorescence. Alternative wavelength such as 780 nm may be used for excitation wavelengths of autofluorescense and 800 to 950 nm may be used for detection. A shorter wavelength range may be used for the fluorescence detection of drug coating transfer confirmation, using one of the combinations of the excitation and emission wavelength shown, for example, in Table 1 shown above. A laser with a wavelength close to the excitation wavelength is chosen and the band pass filter or similar component for the detection near the emission wavelength is used. The laser light of the fluorescent modality for excitation and emission is combined, separated and filtered in the optical combiner/beamsplitter in the PIU. The concentration of the dye in the coating may be adjusted for optimal detection with the bandwidth of the detection filter and the sensitivity of the detector. The two separate signals of fluorescence are used as drug transfer information (the shorter wavelengths) and as tissue characterization (the longer wavelengths for autofluorescense).

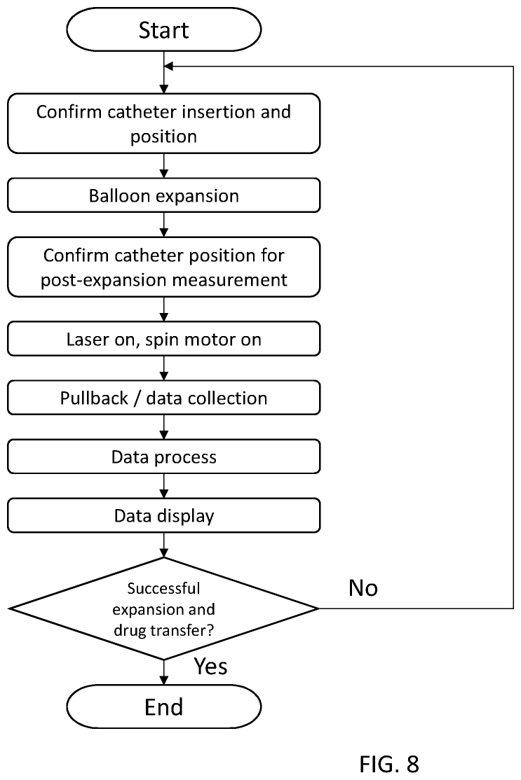

In the workflow of FIG. 8, an exemplary workflow is provided for a catheter inserted into a patient's lumen at a position needing expansion and drug delivery. First the system starts up and then it will ask the user for the confirmation of catheter insertion and its positioning. After confirmation, balloon expansion is done either automatically or manually. The expanded position is held for a time period to allow transfer of drug and fluorescent agent from the outer wall of the balloon to the lumen tissue. Confirmation of position of pullback or data capturing is done next by asking the user to confirm. Once confirmed, the system will turn on the laser, spin motor and system will be ready for pullback data collection. Either by certain timing after the confirmation or by triggering by the user, the pullback is performed. The data is processed and displayed. The user will be prompted to review the data and if the procedure is successful, then the process will end, while if not, the system will confirm for catheter ballooning position and the flow is repeated until the procedure is successful.

### Analysis

In the workflow of FIGS. 5, 7, and 8, the balloon is expanded to release the drug and fluorescent agent and then fluorescence data is collected. This will often occur immediately following the balloon expansion step to obtain information on the amount of drug transferred and released to the tissue. However, additional analysis at a later time point may also be indicated. The clinical safety and efficacy of balloon-delivered drugs depends on drug migration into the arterial (or other) tissue as well as wash-off into the lumen. The drug that is washed off into the lumen will not be present during an imaging catheter scan that is taken soon after the lumen is flushed of blood for OCT and/or fluorescence imaging. As drug migration may occur in a different time domain, sequential or later imaging and analysis of the fluorescence signature over some time after the ballooning process may be indicated. For example, multiple scans over a several minute time frame may be indicated, to estimate how much fluorescence agent and drug are transferred with certainty. The amount of drug transferred may be expressed as concentration of drug, that is the amount (dose) of drug in a unit area or the amount of drug transferred to a measured or predetermined area. The area will correspond to the contact area of the balloon to the vessel inner wall. Processing of data may include, for example, a calculation of dilation of the vessel using the structural imaging (e.g., OCT or IVUS) or physiological analysis (e.g., FFR) to confirm dilation. The processing of data may also include a confirmation of transfer of the drug to the vessel by comparing with a threshold value of average fluorescence value over a predetermined length of the vessel. The processing of fluorescence imaging data may also include calculating the ratio of transferred fluorescence agent with respect to the axial length of the balloon, to show the confirmation of transfer of fluorescence agent and/or drug in lengthwise direction. The fluorescent value may be converted to transferred drug amount or concentration at any step of the processing using lookup table or other correlation coefficients or equations.

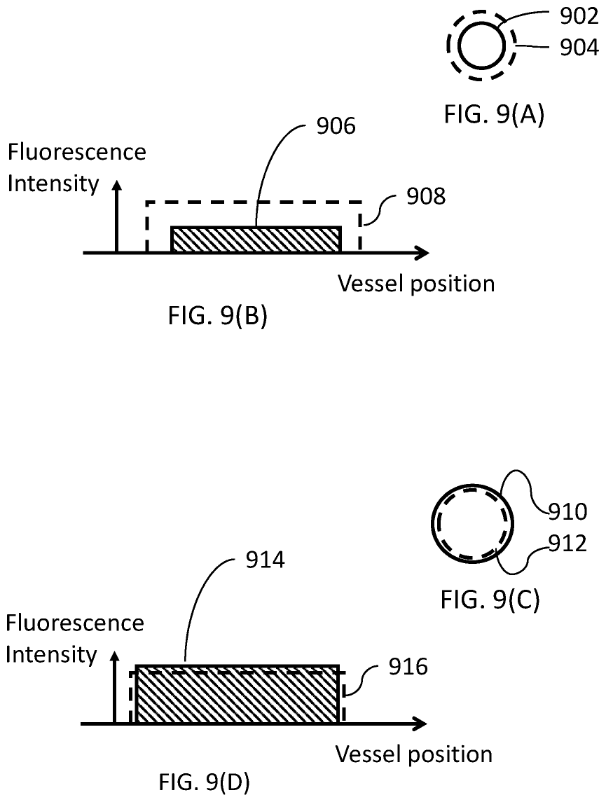

FIGS. 9 (A)-9(D) show an example of graphical representation of the analysis results. In FIG. 9(A), the structural image in the tomographic view 902 shows the lumen imaged to be under expanded compared to the preset, dotted line shown target expansion size 904. FIG. 9(B) is a longitudinal graph that shows the fluorescence value 906 is lower than the targeted length and intensity 908. FIG. 9(C) illustrates a tomographic view 910 with the appropriate expansion representation where the expanded vessel lumen is approximately matching tomographic view and matches the target expansion size 912. The fluorescence intensity in the longitudinal graph FIG. 9(D) is showing fluorescence intensity 914 over the preset target value 916 and the length of the position where the fluorescence is showing up is close to the target area. The target values are shown here for the explanation purposes and it may or may not be presented in the device. It may be up to the physician to decide what the target range is. The required closeness of the values and lengths to the target is a variable which will be determined by other studies, from various practices of physicians, or depending on the balloon, drug and fluorescence agent used, or other parameters. FIGS. 9(A)-(D) show the target values represented with dotted lines (904, 908, 912 and 916), but they may be represented, for example, with a thicker band or a varied color to show tolerance of the target. It may be represented by other expressions of pass/fail, appropriateness or level of expansion of the procedure.

Unless otherwise discussed herein, like numerals indicate like elements. For example, while variations or differences exist between the systems/apparatuses, such as, but not limited to, the system 300, or the systems, catheters, and workflows provided herein. etc. (e.g., differences between the position(s) of the reference reflection 310 (and/or reference arm 304) depending on the OCT and/or fluorescence system or method being used), one or more features thereof may be the same or similar to each other, such as, but not limited to, the light source 302, the various catheters (310, 320, 330, 410, 630) or other component(s) thereof (e.g., the computer 350, etc.). Those skilled in the art will appreciate that the light source 302, the at least one detector 314 and/or one or more other elements of the system 300, may operate in the same or similar fashion to those like-numbered elements of one or more other systems. Those skilled in the art will appreciate that alternative embodiments of the system 300, the catheters, etc. and/or one or more like-numbered elements of one of such systems, while having other variations as discussed herein, may operate in the same or similar fashion to the like-numbered elements of any of the other systems (or component(s) thereof) discussed herein.

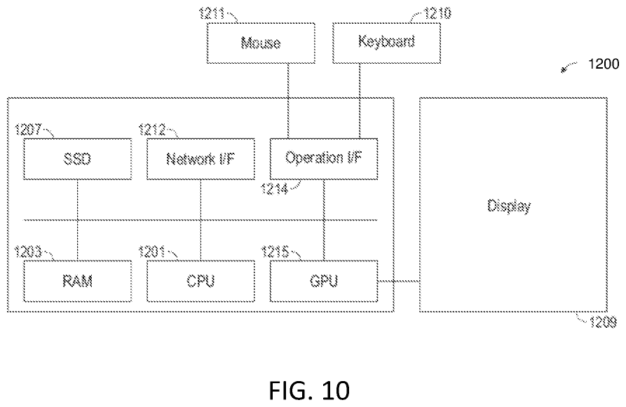

As aforementioned, hardware structure of an embodiment of a computer or console 1200 is shown in FIG. 10. The computer 1200 includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid state drive (SSD) 1207. Preferably, the computer or console 1200 includes one or more display 1209. The computer 1200 may connect with a rotary junction, a motor, the motor PM, the motor SM, and/or one or more other components of a system via the operation interface 1214 or the network interface 1212. The operation interface 1214 is connected with an operation unit such as a mouse device 1211, a keyboard 1210 and/or a touch panel device (not shown). The computer 1200 may include two or more of each component. Alternatively, the CPU 1201 or the GPU 1215 may be replaced by the field-programmable gate array (FPGA), the application-specific integrated circuit (ASIC) or other processing unit depending on the design of a computer, such as the computer 350 or the computer 1200.

The monitor or display 1209 displays the reconstructed image, and may display other information about the imaging condition or about an object to be imaged. The monitor 1209 also provides a graphical user interface for a user to operate a system for example when performing OCT, fluorescence, or other imaging technique(s) or performing an operation of the balloon. An operation signal is input from the operation unit (e.g., such as, but not limited to, a mouse device 1211, a keyboard 1210, a touch panel device, etc.) into the operation interface 1214 in the computer 1200, and corresponding to the operation signal the computer 1200 instructs the system to set or change the imaging condition, and to start or end the imaging. The light source 101 of an OCT sub-system and/or the light source 101 of a fluorescence sub-system as aforementioned may have interfaces to communicate with the computers 1200 to send and receive the status information and the control signals.

The present disclosure and/or one or more components of devices, systems and storage mediums, and/or methods, thereof also may be used in conjunction with various imaging probes. Such probes include, but are not limited to, the OCT imaging systems disclosed in U.S. Pat. Nos. 7,872,759; 8,289,522; and U.S. Pat. No. 8,928,889 to Tearney et al. and arrangements and methods of facilitating photoluminescence imaging, such as those disclosed in U.S. Pat. No. 7,889,348 to Tearney et al., as well as the disclosures directed to OCT and multimodality imaging disclosed in U.S. Pat. No. 9,332,942 and U.S. Patent Publication Nos. 2010/0092389, 2012/0101374, 2016/0228097, as well as in U.S. Pat. Nos. 10,578,422; 10,323,926; 10,558,001; 10,606,064; 10,674,985; 10,743,749; 10,782,117; 10,884,199; 10,895,692; 10,952,616; 11,147,453; and 11,175,126 and in U.S. Patent Publication Nos. 2019/0254506 2019/0313975; 2020/0126195; 2020/0256661; 2020/0345440; 2022-0040454; 2022-0042781; and 2022-0044428 each of which patents, patent publications and patent application(s) are incorporated by reference herein in their entireties.

Although the disclosure herein has been described with reference to particular embodiments, it is to be understood that these embodiments are merely illustrative of the principles and applications of the present disclosure (and are not limited thereto). It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications and equivalent structures and functions.

1. A medical device comprising:
an elongated tube;
a balloon disposed over the elongate tube having at least one drug and at least one fluorescent agent on an outer surface of the balloon;
a probe at the distal end of the elongate tube comprising an optical fiber configured to guide illumination light coming from a light source and an optical member configured for fluorescence imaging; and
one or more detectors configured for fluorescence detection.

2. A medical device comprising:
an elongated tube;
a balloon disposed over the elongate tube having at least one drug and at least one fluorescent agent on an outer surface of the balloon;
a probe at the distal end of the elongate tube,
wherein the probe is configured for fluorescence imaging, and
wherein the probe is configured for structural imaging or physiological sensing;
and
a first detector configured for fluorescence detection; and
a second detector configured for detection of the structural imaging or physiological sensing.

3. The medical device of claim 2, wherein the structural imaging is optical coherence tomography (OCT) imaging and the second detector is configured for detecting an OCT data.

4. The medical device of claim 2, wherein the structural imaging is intravascular ultrasound (IVUS) imaging and the second detector is configured for detecting ultrasound.

5. The device of claim 2, wherein the physiological sensing is fractional flow reserve (FFR) and the second detector is configured for detecting pressure.

6. The medical device of claim 1, wherein the drug and the fluorescent agent are distributed substantially evenly over the outer surface of the balloon.

7. The medical device of claim 1, wherein the drug and the fluorescent agent are a drug-fluorophore conjugate.

8. The medical device of claim 1, further comprising a second fluorescent agent or a second drug.

9. The medical device of claim 1, wherein one of the one or more detectors is configured for detecting tissue autofluorescense.

10. The medical device of claim 1, wherein the probe is configured to rotate within the medical device.

11. A method of analysis comprising:
inserting a catheter comprising an optical probe and a drug-coated balloon into a lumen,
wherein the optical probe comprises an optical fiber configured to guide illumination light coming from a light source and an optical member configured for fluorescence imaging; and
wherein the drug-coated balloon has a drug and a fluorescent agent on the outer surface of the balloon;
expanding the drug-coated balloon in a region of the lumen to create a drug-transfer region of the lumen;
detecting a fluorescence signal from the fluorescent agent in the drug-transfer region with the optical probe;
correlating the fluorescence signal with a concentration of the drug transferred from the drug-coated balloon; and
displaying information based on the concentration of the transferred drug.

12. The method of claim 11, further comprising:
imaging at least a portion of the drug-transfer region of the lumen with the optical probe to obtain an image; and
confirming or verifying dilation of the lumen and/or transfer of drug in the drug-transfer region with the image.

13. The method of claim 12, wherein the imaging is optical coherence tomography image.

14. The method of claim 12, wherein the imaging is intra-vascular ultrasound imaging.

15. The method of claim 11, wherein correlating the fluorescence signal with a concentration of the drug uses a correlation factor obtained from ex vivo studies.

16. The method of claim 10, further comprising detecting FFR.

17. The method of claim 10, further comprising detecting a second fluorescence signal drug-transfer region, wherein the second fluorescence signal is a tissue autofluorescense signal.

18. The method of claim 11, wherein the information based on the concentration of the transferred drug is information that compares the concentration of the drug transferred to a dose of the drug known to be sufficient for a treatment.
